(19) United States
(12) Patent Application Publication
(10) Pub. No.: US 2013/0149723 A1
(43) Pub. Date: Jun. 13, 2013

(54) Method of controlling insect pests in cotton

(71) Applicants: Xi Wang, Central, SC (US); Feng Chen, Central, SC (US)

(72) Inventors: Xi Wang, Central, SC (US); Feng Chen, Central, SC (US)

(73) Assignee: Clemson University, Clemson, SC (US)

(21) Appl. No.: 13/507,238

(22) Filed: Jun. 14, 2012

Related U.S. Application Data
(62) Division of application No. 12/255,940, filed on Oct. 22, 2008, now Pat. No. 7,902,350.
Application No. 12/255,940 is a continuation of application No. PCT/US2007/008512, filed on Apr. 5, 2007.
(60) Provisional application No. 60/689,364, filed on Jun. 10, 2005.

(30) Foreign Application Priority Data
Apr. 5, 2006 (US) PCT/US2007/008512

(51) Int. Cl. C07K 17/10; G01N 33/68

(52) U.S. Cl. CPC C07K 17/10; G01N 33/6803
USPC 435/7.92; 530/391.7; 428/402

(57) ABSTRACT

An assay system is provided in which gossypol is used as a biological marker to detect evolved resistance of insects to Bt cotton. Detection of gossypol using a monoclonal antibody ELISA-based protocol enables at risk populations of insects to be evaluated for evolved resistance to Bt present in a genetically modified cotton.

The specificity of the monoclonal antibody to gossypol also enables the production of nanoparticles having a conjugated monoclonal antibody which retains the ability to selectively bind gossypol. Accordingly, nanoparticles can be provided with additional target ligands, such as antibodies, so as to specifically attach to tumors or cancer cells thereby delivering the gossypol to the target cells.

### RELATED APPLICATIONS

This application claims the benefit of U.S. application Ser. No 60/789,364, filed on 5 Apr. 2006 and which is incorporated herein by reference.

### FIELD OF THE INVENTION

The present invention is directed to a method of growing insect resistant crops in a manner which uses assays of insect pests to monitor the development of possible resistance of pests to genetically modified cotton. The assays are made using a sensitive ELISA protocol which can detect the biological marker gossypol in amounts as low as 5 parts per billion (ppb).

Another aspect of the present invention is directed to a method of treating cancer wherein the therapeutic agent gossypol is bound to a nanoparticle substrate by a gossypol specific monoclonal antibody. The nanoparticle can subsequently provide a binding site for a tumor-specific antibody-directed therapy. Wherein the nanoparticle is a radiographic substrate such as an iron-dextran nanoparticle, the visualization of the tumor-specific binding can be monitored through x-rays or other non-invasive imaging techniques.

### BACKGROUND OF THE INVENTION

Insect resistance to pesticides has been a growing problem in modern agriculture. In addition to resistance to foliar applied insecticides, resistance also occurs with respect to transgenic crops. Transgenic crops are genetically modified to produce a high dose of an inherent toxin capable of killing the insect pest or greatly disrupting the insect's life cycle.

Insect resistance does occur with respect to genetically modified transgenic crops. One strategy to prevent or delay the development of insect resistance to a genetically modified crop includes the use of “refuges”. Refuges are adjacent areas of non-modified crops which may be similar or dissimilar species to the modified transgenic crops. The refuge system has applicability to many pest and disease management techniques. One well known example involves the use of Bt corn which is a hybrid field crop that has been genetically modified to express a toxin in the leaves and stem of the plant. The toxin is a crystal-like protein which naturally occurs in Bacillus thuringiensis and is generally fatal when ingested by the European Corn Borer.

Providing “refuges” of non-Bt crop plants in areas of, or adjacent to, fields of Bt crops to sustain a population of non-resistant individuals, is the recommended Best Management Practice for delaying the onset of resistance to Bt toxins in a variety of insect pests. For instance, with respect to Bt corn, it has been recommended that 5 to 30 percent of acreage planted in corn should be set aside for non-Bt corn. It is further recommended that 40 percent be set aside as a refuge area if the refuge area is to be treated with insecticides.

While the use of a refuge system is well established in certain Bt crops such as corn, the U.S. Environmental Protection Agency is requiring greater evaluation and field testing prior to widespread approval of other genetically engineered crops such as Bt cotton. Field studies require that there be an evaluation of the effectiveness of Bt cotton against target pest insects. In addition, plans require that there be a non-cotton refuge within specified distances of each Bt cotton field to serve as a habitat for susceptible insects. While genetic models and field tests have suggested that such refuge plantings of non-crop plants can significantly slow the evolution of insect resistance to the Bt toxin, specific and detailed information is required to establish the efficacy and long term viability of genetically engineered cotton plants.

Heretofore, there has not been a satisfactory, viable technique to quantify the ratio of insects produced in the Bt cotton versus the non-cotton refuge crop. Until recently, the only method of assessing the relative number of insects produced outside the cotton crop required time consuming field surveys to generate estimates on larvae populations within the crops. The larvae population is used to produce estimates on moth production although it is conceded that extrapolation of moth production from larvae counts is problematic since factors such as predation, parasitism, and soil conditions all have a large impact on pupa survival.

Accordingly, there remains a need to develop assays to determine whether an insect was produced in a Bt cotton or a non-cotton refuge crop. Further, there is a need to develop an assay method that allows assessments of the insect growth and feeding patterns based upon the adult stage of the insect as opposed to a larval form.

### SUMMARY OF THE INVENTION

It is one aspect of at least one of the present embodiments to provide an immunoassay which can detect whether an insect has fed on a Bt cotton as opposed to a non-cotton refuge crop.

It is yet another aspect of at least one of the present embodiments to provide for an enzyme linked immunosorbent assay (ELISA) which uses monoclonal antibodies to measure the presence of a biomarker unique to cotton crops to determine if an insect has fed on a cotton crop as opposed to a non-cotton refuge crop.

It is a further object of the present invention to provide for an ELISA method which can detect a cotton biomarker gossypol at levels as low as about 5 parts per billion (ppb).

It is a further object of the present invention to provide for a process of monitoring insect feeding patterns comprising: growing a first crop having a genetically modified expressed protein, the expressed protein having insecticidal properties, the first crop further containing a biological marker present in at least a portion of a tissue of the first crop; growing a refuge second crop planting in a boundary area in proximity to the first crop; and, assaying test insect populations from at least one of the first crop areas and the refuge second crop areas for the presence of the biological marker within a tissue of the insect.

It is a further object of the present invention to provide for a monitoring process wherein the pest insect population includes insects having a larval stage which may feed on the first crop, the pest insect further having a second developmental non-larval stage in which the pest insect is a winged insect capable of migrating from the first crop into the refuge second crop.

It is a further object of the present invention to provide for a monitoring process where the first crop is a Bt modified cotton and the pest insect is cotton bollworms.

It is an additional aspect of at least one embodiment of the present invention to provide a nanoparticle having an anti-gossypol monoclonal antibody conjugated to the nanoparticle. The conjugated antibody retains the ability to specifically bind the antigen gossypol which has anti-cancer properties. The nanoparticle contains additional binding sites which allow for tumor-specific antibodies or other tumor-specific ligands to be covalently attached to the nanoparticle. In this manner, the resulting nanoparticles may be used as part of an antibody-directed therapy for tumors and cancerous cells.

It is an additional aspect of at least one embodiment of the present invention to provide for a targeted gossypol delivery system in which a monoclonal antibody specific to gossypol is covalently bound to a nanoparticle such as an iron-dextran particle. The conjugated antibody nanoparticle complex allows for the complex to specifically bind gossypol which has demonstrated anti-cancer properties. The nanoparticle contains additional binding sites for tumor-specific ligands including antibodies and other molecules having specificity for a tumor or cancer cell.

It is an additional aspect of at least one embodiment of the present invention to provide for a composition for effecting therapy of a tumor in a patient comprising: a nanoparticle having conjugated thereto an antibody having binding activity directed to gossypol.

It is an additional aspect of at least one embodiment of the present invention to provide for a composition wherein gossypol is bound to the antibody.

It is an additional aspect of at least one embodiment of the present invention to provide for a composition wherein the nanoparticle is an iron-dextran particle.

It is an additional aspect of at least one embodiment of the present invention to provide for a composition wherein the nanoparticle has a size range from about 200 nanometers to about 400 nanometers.

It is an additional aspect of at least one embodiment of the present invention to provide for a composition wherein the nanoparticle has an average size of at least about 225 nanometers and following conjugation with the antibody has an average size of at least about 281 nanometers.

It is an additional aspect of at least one embodiment of the present invention to provide for a composition wherein the nanoparticle further comprises a ligand bound to the nanoparticle, the ligand having a binding activity specific for a target cell.

These and other features, aspects, and advantages of the present invention will become better understood with reference to the following description and appended claims.

### BRIEF DESCRIPTION OF THE DRAWINGS

A fully enabling disclosure of the present invention, including the best mode thereof to one of ordinary skill in the art, is set forth more particularly in the remainder of the specification, including reference to the accompanying drawings.

FIG. 1A is a size distribution of ferromagnetic iron-dextran nanoparticles.

FIG. 1B is a size distribution of ferromagnetic iron-dextran nanoparticles following the covalent attachment of a monoclonal gossypol-antibody.

FIG. 2 sets forth a mechanism of forming the antibody-ferromagnetic iron-dextran conjugates.

FIG. 3 is a transmission electron micrograph of the antibody-ferromagnetic iron-dextran nanoparticles.

FIG. 4 is a graph setting forth the bioactivity of the gossypol-antibody ferromagnetic iron-dextran nanoparticles.

### DESCRIPTION OF THE PREFERRED EMBODIMENT

Reference will now be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

In describing the various figures herein, the same reference numbers may be used throughout to describe the same material, apparatus, or process pathway. To avoid redundancy, detailed descriptions of much of the apparatus once described in relation to a figure is not repeated in the descriptions of subsequent figures, although such apparatus or process is labeled with the same reference numbers.

Cotton plants have long been recognized as unique and valuable sources of fiber, food, and feed. Both the vegetative and reproductive portions of the cotton plant contain dark pigmented glands which are unique to cottonseed. The major component of the pigment glands is free gossypol, a polyphenolic aldehydic compound, which is a unique chemical to distinct cotton crops and non-cotton crops.

Commonly used methods for gossypol analysis include colorimetric AOCS official method (Ba 7-58, 1987; Ba 8-78, 1987) and HPLC method (Abou-Donia and others 1981; Nomeir and Abou-Donia 1982; Hron and others 1990). However, the detection limit for both methods is not sensitive enough to analyze gossypol in 100 mg weight insects. In addition, the AOCS method is time consuming and HPLC requires highly skilled personnel. In accordance with the present invention, there has been developed a monoclonal antibody-based ELISA method which is able to measure gossypol directly, without sample clean up and gossypol derivatization steps required by the HPLC and AOCS methods. The ELISA methods (both direct and indirect ELISA) can detect gossypol as low as 24 ppb (direct), or 5 ppb (indirect) which provide the ability to analyze small samples or samples containing low gossypol content. Additional details on the ELISA protocol and the monoclonal antibody production may be found in reference to the publications entitled, “Monoclonal Antibodies For The Analysis Of Gossypol In Cottonseed Products”, J. Agric. Food Chem., 2004, 52, 709-712 by Xi Wang and Leslie C. Plahak; “Development of Monoclonal Antibody-Based Enzyme Linked Immunosorbent Assay For Gossypol Analysis in Cottonseed Meals”, J. Agric. Food Chem., 2004, 52, 7793-7797 by Xi Wang et al; and “Development of Competitive Direct ELISA For Gossypol Analysis”, J. Agric. Food Chem, 2005, 53, 5513-5517, the disclosures of which are incorporated herein by reference for all purposes.

The ELISA method may be used to measure the presence of gossypol in insects. The presence of gossypol provides an indicator that may be used to monitor the movement of insects between genetically modified cotton and non-genetically modified buffers (refuges), thereby providing valuable pest management information.

The sensitivity of the assays is such that cotton bollworms or tobacco bollworms reared in gossypol containing cotton crops, will contain detectable levels of gossypol at multiple stages of the insect's life cycle. Insects raised on non-cotton crops will not contain gossypol. As such, the presence of gossypol in pest insects is an indicator that the insect was reared in cotton crops and provides valuable information regarding the development of resistance to Bt cotton or other topical insecticides used to combat cotton insect pests.

The present invention provides a novel method for gossypol analysis in individual insects. The method provides information on where an insect was reared, in cotton crops or non-cotton crops. Such data provides valuable information for pest management programs.

### MATERIALS AND METHODS

Materials

Bovine serum albumin (BSA), gossypol, goat anti-mouse peroxidase conjugated IgG+IgM (H+L), Rosewell Park Memorial Institute 1640 (RPMI 1640) were purchased from Sigma Chemical Co. (St. Louis, Mo.). One step ABTS (2,2′-Azino-di-3-ethylbenzthiazoline sulphonate) peroxidase substrate was bought from Pierce (Rockford, Ill.). 61 cotton bollworm larvae were collected in the field, taken to the lab and reared on plant tissue either cotton tissue or non-cotton tissue, in the laboratory at Monsanto Co.. 16 tobacco bollworms (TBW) were reared in the laboratory at Monsanto Co.. Eggs from laboratory susceptible colonies were used. Diet fed TBW were completely reared on Southland Multispecies Lepidopteran diet, and the “cotton-fed” TBW came from larvae which were first reared for 48 hours on a Lepidopteran diet and then reared on cotton squares (buds) in assay cups. The squares were changed every two days until pupation.

Preparation of Culturing Media and is-ELISA Solutions

RPMI cell culture medium, phosphate buffered saline (PBS) and PBST (phosphate buffered saline-tween) solutions were prepared as described previously (Wang and Plhak 2004). Plate coating conjugate, gossypol-BSA (bovine serum albumin), was made through Schiff base formation, and the reaction was performed as described in the previous papers (Wang and Plhak 2004).

Preparation of Monoclonal Antibodies (MAb)

Anti-gossypol monoclonal antibody cell line was growing in 10% FBS in RPMI medium as described (Plhak and Wang, 2004). The tissue culture cell suspensions were collected and centrifuged at 250×g for 5 min to remove the hybridomas. The supernatant was transferred into a beaker and was kept in a cold water bath (0° C.). Ammonium sulfate (35 g/100 ml supernatant) was then slowly (10-15 min) added to obtain 55% saturation with regular stirring and left to stir for another 30 min. The mixture was centrifuged at 9,000×g at 2° C. for 15 min, and the supernatant was discarded and the protein precipitated was resuspended in about 2-4 pellet volumes of PBS buffer. This solution was dialyzed against 3×1500 ml of PBS buffer for 12 hours to remove ammonium sulfate. The protein concentration of the dialyzed solution containing monoclonal antibody was determined using BCA™ Protein Assay Kit (Rockford, Ill.). Following the procedures described by the manufacturer, the dialyzed solution was stored at −20° C. for use in idc-ELISA.

ELISA Protocol.

Indirect competitive ELISA was performed as described as followings: One hundred μL/well of 5 μg/mL of gossypol-BSA in PBS was coated on Immulon® 2 HB microtiter plates (Dynex Technologies, Inc., Chantilly, VA) overnight at 4 ° C. After removing the coating solution by inverting the plate, blocking solution (200 μL/well of 0.5% BSA in PBS) was added and incubated for 30 min at 37 ° C., then the solution was removed and washed with 1×200 PBST. Fifty μL of serially diluted gossypol solutions (100, 10, 1, 0.1, 0.01, 0.001, 0 μg/mL in 10% methanol diluted in PBS) or gossypol extract ( 1/10 dilution in PBS) from insects with 50 μL. of purified monoclonal antibody ( 1/100 dilution in PBS buffer solution) were added. Shaking for 5 min at orbital shaker before incubation for 40 min at 37° C., then the wells were washed using 3×200 μL of PBST, and 100 4/well of goat anti-mouse peroxidase conjugated IgG+IgM (1/10,000 dilution in PBS) was added. After 30 min at 37° C., excess reagents were removed and wells were washed with 3×200 μL of PBST. Then, ABTS substrate (100 μL/well) was added and the absorbance was measured at 405 nm after 30 min in the dark at room temperature on a Biorad Microplate Spectrophotometer.

Sample Preparation

Each insect was ground by pestle in a mortar in the presence of 2 ml of acetone, and the supernatant was transfer into a 15 mL tube. The extractions were performed three times and all the supernatants were pooled together. Then the samples were evaporated to remove the acetone, then 0.5 mL of methanol was used to resolve the extracted gossypol and diluted into 1/10 in PBS. The diluted extract was applied into indirect competitive ELISA as described above for analysis.

Data Analysis

The four parameter sigmoidal curve was used to fit the data (Rodbard 1981).

Y=(A−D)/[1+(X/C)^{B}]+D

Where A is the response at zero concentration of gossypol, D is the response at “infinite” concentration of gossypol, C is the gossypol concentration giving 50% reduction (halfway between A and D, called I₅₀ value), B is the curvature parameter which determines the steepness of the curve, X is gossypol concentration, and Y is the corresponding absorbance. The gossypol standard curve was obtained by plotting Log10 of standard gossypol concentration against the absorbance. Each test concentration was performed triplicate for standard and three replicates for samples. Each plate includes its own gossypol standard curve, and absorbance from sample was interpolated on the curve performed in the sample plate. The limit of detection was defined as 10% inhibition of the color (Skerritt 1995). All the absorbance higher than 90%Amax will be the cutoff data to define the negative (non-cotton crop reared insects) and positive samples (cotton crop reared insects).

### RESULTS AND DISCUSSION

Gossypol Analysis in Insects.

ELISA results for insect analysis showed that 97% of the samples tested positive are from cotton crops and 100% of tested negative are from non-cotton crops, indicating that this ELISA protocol is a good tool to determine whether the insect was hosted in cotton crops or non-cotton crops. Such a tool is useful for pest control management.

The sensitivity of the ELISA assay allows for a cost effective, rapid screening protocol for detecting evolved resistance to Bt cotton from insect pests. Heretofore, the analytical techniques for detection of evolved resistance to Bt cotton has lacked either sensitivity, cost effectiveness, or ease of use. The rapid screening protocol requires no pre-treatment or processing of the biological samples other than the direct sampling of the insect ground supernatant.

The screening protocol thereby allows a process of monitoring field grown insects to determine whether the insects have fed on a Bt-cotton crop. The gossypol present in Bt cotton crops provides a biological marker which makes it possible to determine if insects collected in the field have fed on the Bt cotton. Accordingly, insects can be screened from samples collected within the Bt cotton planting, samples may be screened from adjacent refuge crop species, and through subsequent analysis make a determination as to whether Bt resistance is occurring in insects. For instance, if gossypol is detected in insects obtained within a refuge area, it is an indication that the insects fed on the Bt cotton but survived. Through population sampling and statistical analysis, the extent of resistance that may have developed can be determined. Accordingly, additional pest management controls may be implemented to delay or suppress the spread of the resistant insects.

Certain insect pests may feed on cotton in a larval form and then undergo metamorphosis into a more mobile (winged), adult insect stage. The sensitivity of the assay is such that residual gossypol present in the adult insect body may be detected, even if the gossypol was ingested in an earlier life cycle stage. Accordingly, to the extent a larval form was feeding on Bt cotton and, in a subsequent adult life cycle stage migrated into a refuge area, such evolved resistance can be detected. There is sufficient residual gossypol in the insect's body that the described ELISA assay can detect the presence of the biological marker gossypol even after the insect has migrated to a different food source and feeding habits.

An additional aspect of the monoclonal antibody directed to gossypol involves the ability to provide tumor-specific therapeutic agents for the treatment and/or neutralization of cancer and tumor cells. Targeting drug delivery into the specific site with rapid and specific drug accumulation has become one of the most important aspects for cancer chemotherapy. The concept of drug delivery using magnetic nanoparticles greatly benefits from the fact that nanotechnology has developed to a stage that makes it possible not only to produce magnetic nanoparticles in a various size distribution but also to engineer a particle having a surface to provide a site specific for drug delivery. Gossypol, a phenolic compound, has shown suppression activity on a variety of cancer cell lines. In accordance with this invention, it is possible to construct dextran magnetic nanoparticles in which the anti-gossypol antibody is conjugated to the nanoparticle. The anti-gossypol dextran magnetic nanoparticles may be used for a rapid detection of gossypol or for delivery of gossypol for cancer treatment to a population of targeted cells.

### MATERIAL AND METHODS

Sephacryl S-300, FeCl₂.4H₂O, acetic acid, and dialysis tubing (Nominal MWCO 6,000-8,000) were bought from Fisher Scientific (Atlanta, GA). Bovine serum albumin (BSA), Tween 20, NaIO₄, FeCl₃.6H₂O, NaBH₄, ammonium sulfate, goat anti-mouse peroxidase conjugated IgG+IgM (H+L), and gossypol were purchased from Sigma Chemical Co. (St. Louis, Mo). One step ABTS (2,2′-Azino-di-3-ethylbenzthiazoline sulphonate) peroxidase substrate, ImmunoPure (Protein A) IgG Purification Kit , and ImmunoPure Horseradish Peroxidase (HRP) were bought from Pierce (Rockford, Ill.). Dextran-40 was bought from VWR International Inc. (Pittsburgh, Pa.). Immulon® 2 HB microtiter plates were from Dynex Technologies, Inc. (Chantilly, Va.).

Synthesis of Magnetic Iron-Dextran Nanoparticles.

Magnetic iron-dextran particles were prepared as described (Molday and Mackenzie, 1982) with minor modifications. Briefly, 1.5 g of dextran, 0.234 g of FeCl₃.6H₂O and 0.086 g of FeCl₂.4H₂O were dissolved in 3 mL of distilled water, and the pH of the reactant mixture was adjusted to 10-11 by gradually adding 3 mL of 7.5% (v/v) NH₄OH while stirring. After incubation of 30 min at 70 ° C., the mixture was neutralized by adding 10% acetic acid.

Aggregates were removed by centrifugation at low speed (2,000 rpm) for 10 min, and the supernatant was collected and dialyzed nominal MWCO 6,000-8,000 against deionized water for 24 hr at 4 ° C. with water change every four hours. The formed ferromagnetic iron-dextran particles were separated from unbound dextran by gel filtration chromatography on Sephacryl S-300 (2.0 cm×40 cm) column eluted with 10 mM of phosphate buffer (pH7.2). The concentration of the purified ferromagnetic iron dextran particles was determined by dry weight analysis.

Conjugation of Antibody with Dextran Ferromagnetic Iron Nanoparticles.

Anti-gossypol monoclonal antibody was conjugated to the magnetic iron dextran particles by the periodic oxidation-borohydride reduction procedures modified from Nakene and Kawaoi (1974). Two mL of dextran iron particles from above (8 mg/mL) were oxidized with 0.5 mL of 0.05 M of NaIO4. After stirring for 1 hr at room temperature, the solution was dialyzed (MWCO 6,000-8,000) against distilled water overnight at 4° C. Then, 1 mL of Protein A affinity column purified anti-gossypol monoclonal antibody (1.5 mg/mL) (Wang et al., 2005) was added, and incubated for 24 hr at 4° C. The products were stabilized with 0.5 mL of 0.5 M reducing reagent NaBH₄ for 2 hr at 4° C. Then the solution was dialyzed against 10 mM phosphate buffer (pH7.2) overnight at 4° C. The conjugates were separated from unbound antibody by gel filtration chromatography on Sephacryl S-300 (2.0 cm×40 cm) column eluted with 10 mM phosphate buffer (pH7.2).

Particle Size Distribution.

The average particle size and the size distribution before and after conjugation with anti-gossypol monoclonal antibody was determined using Coulter N4 Plus Particle Sizer (Beckman). Nanoparticle solution from above was diluted with distilled water and determined at a detector angle of 90°, a wavelength of 633 nm, a refractive index of 1.333, temperature of 25° C., and a running time 180 sec.

Transmission Electron Microscopy (TEM).

Nanoparticles before and after antibody conjugation were placed on to copper grid and examined by a Hitachi HD-2000 TEM/STEM system equipped with a CCD camera for digital imaging.

Assay of Bioactivity of the Antibody-Ferromagnetic Iron-Dextran Conjugates by Competitive Indirect ELISA.

The indirect competitive ELISA (Wang et al., 2004) was modified to determine the bioactivity of monoclonal antibody after conjugation with ferromagnetic iron dextran particles. 100 μL/well of 10 μg/mL gossypol-BSA conjugate was added to an Immulon® 2 HB microtiter plate and incubated overnight at 4° C. After removing the coating solution, blocking solution (200 μL/well of 1% BSA in PBS) was added and incubated at 37 ° C. for 30 min. Unbound materials were washed away with 3×200 μL of PBST, 50 μL/well of serially diluted gossypol standards (100, 10, 1, 0.1, 0.01, 0.001, and 0 μg/mL of gossypol in 10% methanol) were added, immediately followed by adding 50 μL/well of gossypol antibody-ferromagnetic iron-dextran conjugates from above (¼ dilution with PBS). After incubation 45 min at 37 ° C., the solution was washed with 3×200 μL of PBST, and the gossypol antibody activity was tracked by adding 100 μL/well of 1/10,000 diluted goat anti-mouse peroxidase conjugated IgG+IgM in PBS. Followed 45 min incubation at 37° C., plates were then washed with 3×200 μL of PBST and ABTS substrate solution (100 μL/well) was added, and the absorbance at 405 nm was measured after 30 min on microplate spectrophotometer (Biorad, Calif.). I₅₀ value (gossypol concentration casing 50% reduction) was determined based on the least square errors of the observed data in a four parameter equation.

### RESUTS AND DISSCUSSION

Synthesis of Antibody Conjugated Magnetic Iron-Dextran Nanoparticles.

Magnetic iron dextran nanoparticles were produced by chemical co-precipitation of Fe (II) and Fe (III) chloride in alkaline condition to produce macromolecule dextran coated iron (Fe₃O₄, and/or Fe₂O₃) nanoparticles.

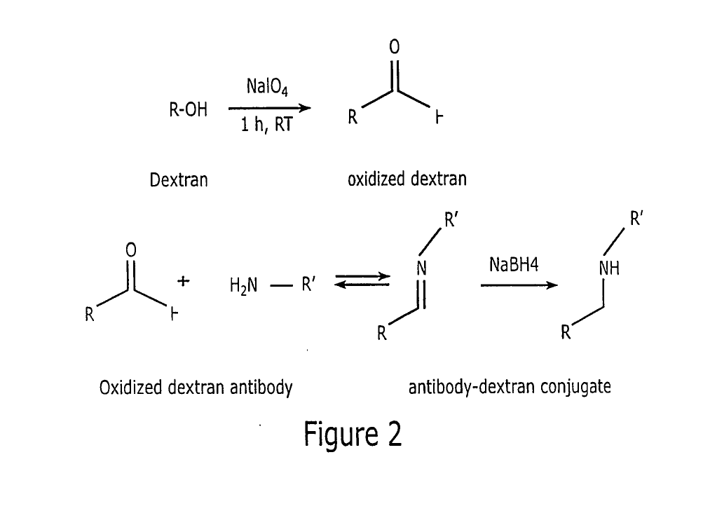

Macromolecule dextran plays a critical role to serve as the biofunctional coating material. These particles have particularly interesting characteristics such as easy preparation, chemical stability, and quantitative controlling of their multiple functionalization (Templeton et al., 2000). Dextran coated particles provide —OH groups on the surface of the particle, so the magnetic iron-dextran nanoparticles could be oxidized by NaIO₄ to introduce carbonyl groups into the dextran nanoparticles. Subsequently, the carbonyl groups reacted with the E-amino group of the anti-gossypol antibody to form antibody-magnetic iron dextran nanoparticle conjugate which was stabilized by adding reducing agent NaBH₄ as seen in FIG. 2.

Characterization of Antibody Conjugated Magnetic Iron-Dextran nanoparticles.

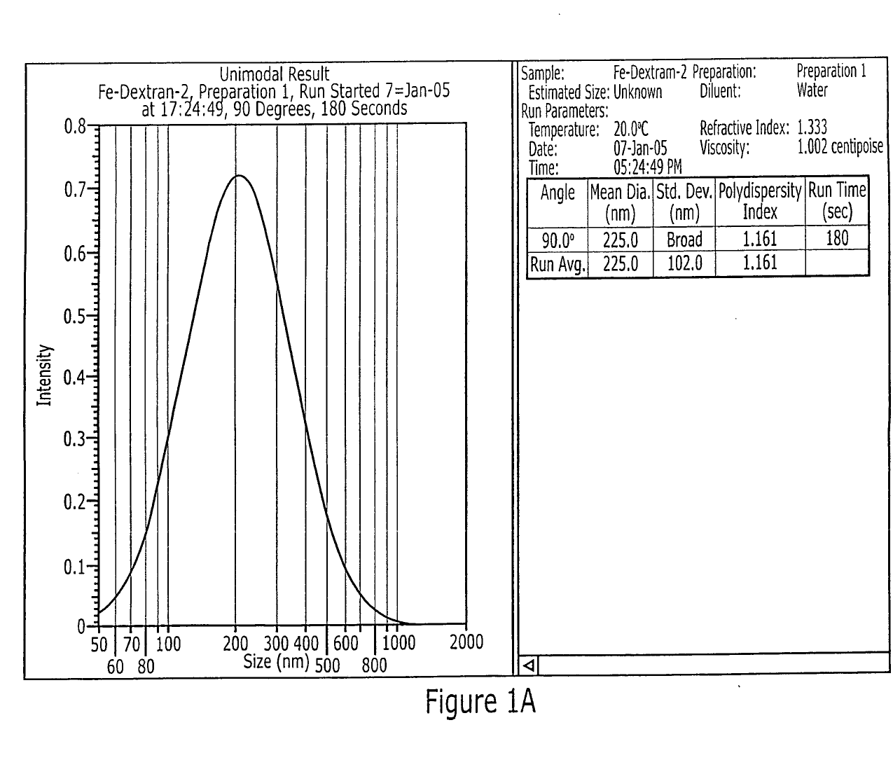

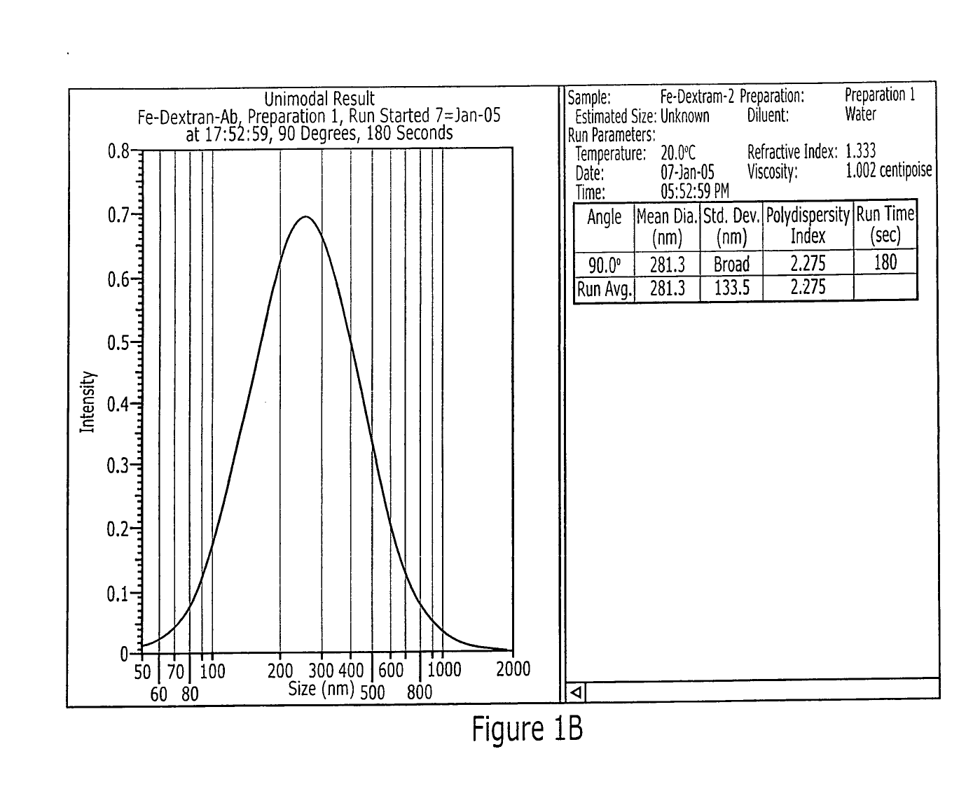

The size and distribution of magnetic iron-dextran nanoparticles and antibody conjugated nanoparticles were determined. The magnetic nanoparticle sizes (or size distribution) are influenced by the ratio of reactants, pH, mixing rate, etc. (Li et al., 1996). The average size of ferromagnetic particles is 225 nm under the reaction condition in this study (FIG. 1A). Increasing the molar ratio between iron:dextran by 25% could increase the nanoparticle size to about 400 nm (data not shown). After monoclonal antibody was conjugated with ferromagnetic iron-dextran particles, the average size of the particles increased from 225 nm to 281 nm in diameter (FIG. 1B). Based on the size of protein (antibody) 5-50 nm, there may be more than one antibody covalently bound with dextran particles, resulting in a size increase of about 56 nm. These magnetic iron particles and antibody conjugated nanoparticles were stable in phosphate buffer (pH 7.2) and there was no apparent aggregates appeared after three months storage at 4° C.

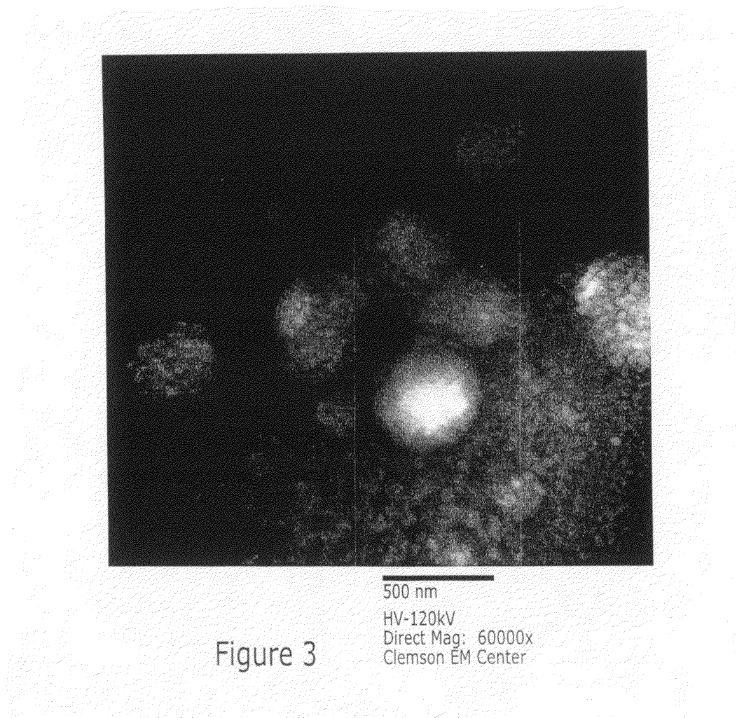

The TEM results showed that the ferromagnetic iron-dextran particles (data not shown) and antibody-ferromagnetic iron-dextran particles were in spherical shape (FIG. 3).

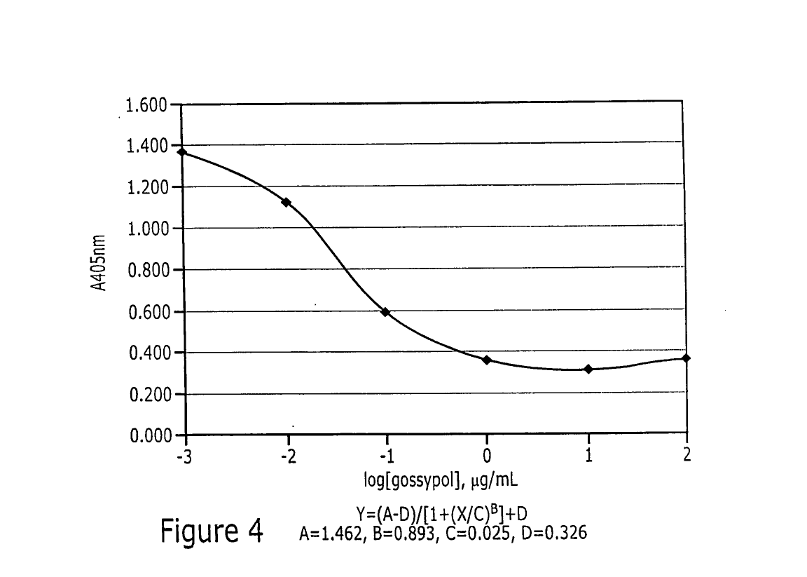

The indirect competitive ELISA results showed anti-gossypol antibody can be successfully conjugated to the prepared iron-dextran nanoparticles. Also, this antibody-nanoparticle conjugate possesses the essential biofunctions to capture antigen gossypol. Under the test conditions, the detection limit (defined as 10% of the color inhibition) can reach to 25 ppb (FIG. 4).

Besides the analytical application, this antibody-magnetic nanoparticles concept can be applied into other fields. If nanoparticles were conjugated with groups that permitted specific recognition of cell types, a more precise localization of selected cells could be achieved (Molday and Mackenzie, 1982; Rembaum, 1984). This active targeting is based on the use of ligands that can bind to a protein, for example, a cell surface receptor. If the magnetic nanoparticles were conjugated with an anti-drug antibody (e.g. anti-gossypol antibody), a specific anti-cancer antibody or other ligand can also be conjugated and used to attach the nanoparticles to cancer cells. (Ito, 2004; Liabakk, 1990). Similar methodology can be used to incorporate an anti-virus antibody which could bond the nanoparticle to a virus, or with an anti-bacterial antibody which could attach the nanoparticle to bacteria. The presence of magnetic iron as part of the nanoparticle would facilitate the separation of bound bacteria from the matrix (Pyle et al., 1999). Additional methods and uses of targeted therapies using antibodies may be seen in reference to U.S. Pat. No. 7,011,812, entitled “Targeted Combination Immunotherapy of Cancer and Infectious Diseases”, assigned to Immunomedics, Inc. (Morris Plains, NJ), and which is incorporated herein by reference.

Additionally set forth below are references which have been cited in the application. These references are incorporated herein by reference in their entirety for all purposes.

### REFERENCES

Abou-Donia, S. A.; Lasker, J. M.; Abou-Dunia, M. B. High performance liquid chromatographyic analysis of gossypol. J. Chromatogr. 1981, 206, 606-610.
AOCS. Free gossypol. Ba 7-58. 1987. In Official Methods and Recommended Practices of the American Oil Chemists' Society. American Oil Chemists' Society, Champaign, Ill.
AOCS. Total gossypol. In Official Methods and Recommended Practices of the American Oil Chemists' Society. 1987. American Oil Chemists' Society, Champaign, Ill. Ba 8-78.
Calhoun, M. C.; Huston, J. E.; Baldwin, B. C. Jr.; Kuhlmann, S. W.; Engdahl, B. S.; Bales, K. W. Effects of cottonseed meal source and dietary crude protein on performance of early-weaned lambs: with observations on gossypol toxicity. Sheep and Goat, Wool and Mohair, 1990 Research Reports. PR 4790, Texas A & M University System, College Station, Tex. 1990.
Calhoun, M. C.; Huston, J. E.; Kuhlmann, S. W.; Baldwin, B. C. Jr.; Engdahl, B. S.; Bales, K. W. Comparative toxicity of gossypol acetic acid and free gossypol in cottonseed meal and pima cottonseed to lambs. Sheep and Goat, Wool and Mohair, 1990 Research Reports. PR 4779, Texas A & M University System, College Station, Tex. 1990.
Calhoun, M. C. Safe levels of cottonseed for cattle. In Cotton Incorporated Agricultural Research Reports. Summary Reports 1996, Cotton Incorporated, Raleigh, N.C. 1996.
Chen, T.; Dwyre-Gygax, C.; Smith, R. S.; Breuil, C. Enzyme linked immunosorbent assay for didecyldimethylammonium chloride, a fungicide used by the forest products industry. J. Agric. Food Chem. 1995, 43, 1400-1406.
Cho, Y. A.; Kim, Y. J.; Hammock, B. D. Lee, Y. T.; Lee, H. S. Development of a microtiter plate ELISA and a dipstick ELISA for the determination of the organophosphorous insecticide fenthion. J. Agric. Food Chem. 2003, 51, 7854-7860.
Eagle, E.; Davies, D. L. Effect of long-term storage on acute oral toxicity and gossypol content of cottonseed pigment glands. J Am Oil Chem Soc. 1958, 35, 36-37.
Eagle, E.; Bialek, H. F.; Davies, D. L.; Bremer, J. W. Biological vs. chemical evaluation of toxicity and protein quality of cottonseed meals. J. Am. Oil Chem. Soc. 1956, 33, 15-20.
González-Garza, M. T.; Matlin, S. A.; Mata-Cardenas, B. D.; Said-Fernandez, S. S. Entamoeba histolytica: Inhibition of malic enzyme and alcohol dehydrogenase by (+/−)-, (+)-, and (−)-gossypol. Arch. Med. Res. 1993, 24, 183-187.
Hammock, B. D.; Gee, S. J.; Harrison, R. O.; Jung, F.; Goodrow, M. H.; Li, Q. X.; Lucas, A. D.; Szkacs, A.; Sundaram, K. M. Immunological technology in environmental analysis: addressing critical problems. In Immunological Methods for Enviromental analysis. Van Emon, J. M., Mumma, R. O. Eds. ACS Symposium Series 442; American Chemical Society: Washington, D.C. 1990; pp.112-139.
Hron, R. J. Sr.; Kuk, M. S.; Abraham, G. Determination of free and total gossypol by high performance liquid chromatography. J. Am. Oil. Chem. Soc. 1990, 67, 182-187.
Lane, C. F. Sodium cyanoborohydride—a highly selective reducing agent for organic functional groups. Synthesis March. 1975, 135-146.
Lin, T. S.; Schinazi, R. F.; Zhu, J.; Birks, E.; Carbone, R.; Si, Y.; Wu, K.; Huang, L.; Prusoff, W. H. Anti-HIV-1 activity and cellular pharmacology of various analogues of gossypol. Biochem. Pharmacol. 1993, 46, 251-255.
Markman, A. L. Gossypol in cotton plant. content and localization. In Gossypol and its derivatives. Markan, A. L., Rzhekhin, V. P. Eds. The U. S. Department of Agriculture and the National Science Foundation, Washington, D C. 1968; pp. 1-9.
Matlin, S. A.; Zhou, R.; Bialy, G.; Blye, R. P.; Naqvi, R. H.; Lindberg, M. C. (−)-Gossypol: an active male antifertility agent. Contracept. 1985, 31(2), 141-149.
Menaul, P. The physiological effect of gossypol. J. Agric. Res. 1923, 26, 233-237.
Mencius, J. J.; Montoya, A. Development of an enzyme-linked immunosorbent assay for 3,5,6-trichloro-2-pyridinol. 2. Assay optimization and application to environmental water samples. J. Agric. Food Chem. 1996, 44, 3710-3716.
Meulenberg, E. P.; Mulder, W. H.; Stocks, P. G. Immunoassays for pesticides. Environ. Sci. Technol. 1995, 29, 553-561.
Nomeir, A. A.; Abou-Donia, M. B. Gossypol: high-performance liquid chromatographic analysis and stability in various solvents. J. Am. Oil Chem. Soc. 1982, 59, 546-549.
Rodbard, D. Mathematics and statistics of ligand assays: an illustrated guide. In Ligand assay. Langan, J., Clapp, J. J. Eds. Masson Publishing, New York. 1981; pp. 45-101.
Sherry, J. P. Environmental chemistry: the immunoassay option. Crit. Rev. Anal. Chem. 1992, 23, 217-300.
Skerritt, J. H. Analytical aspects of immunoassay of agrochemicals. In New frontiers in agrochemical immunoassay. Kurtz, D. A., Skerritt, J. H., Stanker, L. Eds. AOCS International: Arlington, Va. 1995; pp. 1-16
Wang, N. G.; Zhou, L. F.; Guan, M. Z.; Lei, H. P. Effect of (−)- and (+)-gossypol on fertility in male rats. J. Ethnopharm. 1986, 20, 21-24.
Wang, X.; Plhak, L. C. Production, characterization and application of anti-gossypol polyclonal antibodies. J. Agri. Food Chem. 2000, 48, 5109-5116.
Wang, X.; Plhak, L. C. Monoclonal antibodies for the analysis of gossypol in cottonseed products. J. Agri. Food Chem. 2004, 52, 709-712.
Wang, X.; Chen, F., Wan, P. J.; Huang, G. H. Enzyme linked Immunosorbent Assay for Gossypol Analysis in Cottonseed Meals. J. Agri. Food Chem. 2004, 52, 709-712.
Babincova, M.; Leszczynska, D.; Sourivong, P.; Babinec, P. Selective treatment of neoplastic cells using ferritin-mediated electromagnetic hyperthermia. Med Hypotheses. 2000, 54(3):177-179.
Farokhzad, O. C.; Jon, S.; Khademhosseini, A.; Tran T N.; Lavean D A.; Langer, R. Nanoparticle-aptamer bioconjugates: a new approach for targeting prostate cancer cells. Cancer Res. 2004, 64: 7668-72.
Ito, A.; Kuga, Y.; Honda, H.; Kikkawa, H.; Horiuchi, A.; Watanabe, Y.; Kobayashi, T. Magnetite nanoparticle-loaded anti-HER2 immunoliposomes for combination of antibody therapy with hyterthermia. Cancer Letters. 2004, 212:167-175.
Kumar, M. N. V.; Nano and microparticles as controlled drug delivery devices. J. Pharm. Pharmaceut. Sci. 2000, 3:234-258;
Lambert, G. Fattal, E. Pinto-Alphandary, H. Gulik, A. Couvreur, P. Polyisobutylcyanoacrylate nanocapsules containing an aqueous core for the delivery of oligonucleotides. Int J Pharm. 2001. 214, 13:16.
Li, M. Q.; Xu, H. X.; Zuo, J.; Ji, A.; He, B. L.; Pang, Y.; Huang, J. Y.; Niu, R. F. Preparation of dextran immunological magnetic nanoparticles and their application to combined targeting carrier (in Chinese). Science in China Series B-Chemistry. 1996, 26: 193-198.
Liabakk, N. B.; Nustad, K.; Espevik, T. A rapid and sensitive immunoassay for tumor necrosis factor using magnetic monodisperse polymer particles. J. Immunol. Methods, 1990, 134(2): 253-259.
Liu, C. G. Desai, K. G. Chen, X. G. Park, H. J. 2005. Linolenic acid-modified chitosan for formation of self-assembled nanoparticles. J Agric Food Chem.53, 437-441.
Moghimi, S. M.; Hunter, A. C.; and Murray, J. C. Long-circulating and target-specific nanoparticles: theory to practice. Pharmacol. Rev. 2001, 53:283-318;
Molday, R. S.; Mackenzie, D. Immunospesific ferromagnetic iron-dextran reagents for the labeling and magnetic separation of cells. J. Immunol. Methods, 1982, 52:353-367.
Moore, A.; Marecos, E.; Bogdanov, A., Jr.; Weissleder, R. Tumoral distribution of long-circulating dextran-coated iron oxide nanoparticles in a rodent model. Radiology 2000, 214: 568-574.
Nakane, P. K. and Kawaoi, A. Peroxidase-labeled antibody. A new method of conjugation. J Histochem Cvtochem. 1974, Dec.; 22(12):1084-91.
Pinto-Alphandary, H.; Andremont, A.; and Couvreur, P. Targeted delivery of antibiotics using liposome and nanparticles: research and applications. Int. J. Antimicrob. Agents, 2000, 13:155-168.
Pyle, B. H., S. C. Broadaway, and G. A. McFeters. Sensitive detection of Escherichia coli O157:H7 in food and water by immunomagnetic separation and solid-phase laser cytometry. Appl. Environ. Microbiol. 1999, 65:1966-1972.
Rembaum, A. Cell labeling with magnetic and non magnetic immunomicrospheres for separation and diagnosis. Pure Appl. Chem., 1984, 56(10):1305-1307.
Templeton, A. C.; Wuelfing, W. P.; and Murray, R. W. Monolayer-protected cluster molecules. ACC. Chem. Res. 2000, 33:27-36.
Wang, J; Chen, F.; Wang, X; Wan, P. J.; He, G.; Li, Z. Development of a competitive direct ELISA for gossypol analysis. J. Agri. Food Chem. 2005.
Wang, X. and Plhak, L. C. 2004. Monoclonal antibodies for the analysis of gossypol in cottonseed products. J. Agric. Food Chem. 52: 709-712.

Wang, X.; Chen, F.; Wan, P. J.; Huang, G. Development of monoclonal antibody-based enzyme-linked immunosorbent assay for gossypol analysis in cottonseed meals. J. Agri. Food Chem. 2004, 52, 7793-7797.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention which is set forth in the following claims. In addition, it should be understood that aspects of the various embodiments may be interchanged, both in whole, or in part. Therefore, the spirit and scope of the appended claims should not be limited to the description of the preferred versions contained therein.

That which is claimed:

1. The process of monitoring insect feeding patterns comprising:
growing a first crop having a genetically modified expressed protein, said express protein having insecticidal properties, said first crop further containing a biological marker present in at least a portion of a tissue of said first crop;
growing a refuge second crop planting in a boundary area in proximity to said first crop; and,
assaying insect populations from at least one of said first crop area or said refuge second crop area for the presence of said biological marker within a tissue of said insect.

2. The process according to claim 1 wherein said assaying step is an ELISA assay.

3. The process according to claim 2 wherein said ELISA assay is an indirect assay having a sensitivity of at least about 5 ppb.

4. The process according to claim 1 wherein said insect population includes insects having a larval stage which may feed on the first crop, said insect further having a second developmental non-larval stage in which said insect is a winged insect capable of migrating from said first crop into said refuge second crop.

5. The process according to claim 1 wherein said first crop is a Bt modified cotton and said insect is a cotton bollworm.

6. The process according to claim 5 wherein said step of assaying said insect populations further includes assaying insects at a non-larval stage of development.

7. The process according to claim 1 wherein said biological marker is gossypol and said first crop is Bt cotton.

8. The process according to claim 6 wherein said step of assaying said insect populations further includes assaying insects for the biological marker gossypol.

9. The process according to claim 4 wherein said step of assaying insect populations further includes assaying insects collected from said refuge second crop.

10. The process of monitoring insect populations for evolved resistance to at least one of a pesticide or an expressed protein having insecticidal properties comprising:
growing a first crop having a biological marker present in at least a portion of a tissue of said first crop, said first crop having insect control treatment consisting of at least one of a pesticide or an expressed protein within said first crop;
growing a refuge second crop planting in a boundary area in proximity to said first crop; and,
assaying insect populations from said refuge second crop planting for the presence of said biological marker within a tissue of said insect.

11. The process according to claim 10 wherein said assaying step is an ELISA assay.

12. The process according to claim 10 wherein said insect population includes insects having a larval stage which feeds on the first crop, said insect further having a second developmental non-larval stage in which said insect is a winged insect capable of migrating from said first crop into said refuge second crop.

13. The process according to claim 10 wherein said biological marker is gossypol and said first crop is Bt cotton.

14. The process according to claim 10 wherein said expressed protein is Bt.

15. A composition for effecting therapy of a tumor in a patient comprising:
a nanoparticle having conjugated thereto an antibody having binding activity directed to gossypol.

16. The composition according to claim 15 wherein gossypol is bound to said antibody.

17. The composition according to claim 15 wherein said nanoparticle is an iron-dextran particle.

18. The composition according to claim 17 wherein said nanoparticle has a size range from about 200 nanometers to about 400 nanometers.

19. The composition according to claim 17 wherein said nanoparticle has an average size of at least about 225 nanometers and following conjugation with said antibody has an average size of at least about 281 nanometers.

20. The composition according to claim 15 wherein said nanoparticle further comprises a ligand bound to a surface of said nanoparticle, said ligand having a binding activity specific for a target cell.
